(19) United States
(12) Patent Application Publication
(10) Pub. No.: US 2023/0381267 A1
(43) Pub. Date: Nov. 30, 2023

(54) OXYTOCIN TREATMENT FOR HYPERMOBILE EHLERS-DANLOS SYNDROME

(71) Applicant: Baylor College of Medicine, Houston, TX (US)

(72) Inventor: Brendan Lee, Houston, TX (US)

(21) Appl. No.: 18/027,340

(22) PCT Filed: Sep. 29, 2021
(86) PCT No.: PCT/US2021/071645

§ 371 Date: Mar. 20, 2023

Related U.S. Application Data
(60) Provisional application No. 63/085,740, filed on Sep. 30, 2020.

(51) Int. Cl. A61K 38/095; A61P 19/04

(52) U.S. Cl. CPC A61K 38/095; A61P 19/04

(57) ABSTRACT

Embodiments of the present disclosure concern the treatment and/or prevention of Ehlers-Danlos Syndrome (EDS) or its manifestations using therapeutic compositions, such as oxytocin and/or oxytocin analogues. The therapeutic compositions may decrease symptoms and/or reverse disease progression.

This application claims priority to U.S. Provisional Patent Application Ser. No. 63/085,740, filed Sep. 30, 2020, which is incorporated by reference herein in its entirety.

### BACKGROUND

### I. Technical Field

This disclosure relates at least to the fields of molecular biology, cell biology, physiology, and medicine.

### II. Background

Hypermobile Ehlers Danlos Syndrome (EDS) is a multisystemic condition involving mainly connective tissues such as joints and skin but may also involve other systems such as the cardiovascular, gastrointestinal, autonomic, and neurological systems. The diagnosis of this condition is based on clinical criteria only and, to date, no molecular etiology has been identified. The major component of this condition is generalized joints hypermobility presenting with high Beighton score (acceptably reliable assessment tool for generalized hypermobile joints), joint pain and joint instability and dislocations. Other clinical manifestations may include abnormal scar formation (not as severe as in ‘classical type’ EDS), cardiac structural abnormalities (mitral valve prolapse and aortic root dilatation), postural orthostatic tachycardia syndrome (POTS), chronic fatigue, atopy, and more. One of the major clinical features that accompany this condition is pain. Pain is usually chronic and can be generalized and/or localized. Pain usually involves the musculoskeletal system with joints and back pain but can involve other systems such as abdominal pain and headache. Neuropathic pain is also commonly reported in this population of patients. The current approaches to pain management, including physical therapy and pain medications, are suboptimal as many individuals continue to have chronic pain with these measures. Thus, new treatment approaches that would help manage pain in this population would be important to affected individuals.

The involvements of oxytocin in modulating chronic pain in humans was also studied in the past. Effect of oxytocin on chronic pain was evaluated in multiple conditions including irritable bowel disease, chronic constipation, fibromyalgia, and tension-type headache and migraine. Only the study to measure oxytocin effect on irritable bowel syndrome was using IV administration of oxytocin (while all others used intranasal administration). This study showed a significant effect of oxytocin on pain tolerance. Intranasal OXT was reported to relieve headache in a dose-dependent manner in individuals with tension-type headache and migraine.

Postural orthostatic tachycardia syndrome (POTS) is the most common of several types of dysautonomia, characterized by dysfunction of the autonomic nervous system manifesting with symptoms of orthostatic intolerance with or without associated orthostatic hypotension and excessive autonomic excitation. Given the numerous presenting musculoskeletal symptoms of POTS and its known associations with other clinical entities like Ehlers-Danlos syndrome, POTS constitutes an unusual treatment challenge of which the orthopaedic surgeon and other related healthcare providers should be aware.

### BRIEF SUMMARY

Certain embodiments of the disclosure encompass methods for treating, preventing, ameliorating, or reducing one or more symptoms, diseases, syndromes, and/or disorders, such as chronic musculoskeletal pain, Hypermobile Ehlers-Danlos syndrome, Dysautonomia spectrum disorder, and/or postural orthostatic tachycardia syndrome. In some embodiments, Hypermobile Ehlers-Danlos syndrome comprises Ehlers-Danlos syndrome type III. The disease may be mild, moderate, or severe.

In some embodiments, a therapeutically effective amount of one or more compositions is administered to an individual, such as an individual suffering from any disease, syndrome, and/or disorder encompassed herein. In some embodiments, the composition comprises oxytocin and/or an analogue of oxytocin. The analogue of oxytocin may comprise carbetocin, demoxytocin, Z-prolyl-D-leucine, 1-N-methylhemicystine-oxytocin, 1-D-hemicystineoxytocin, glycyl-, leucyl-, phenylalanyl-, prolyl-, glycyl-glycyl-, leucyl-leucyl, or leucyl-glycyl-glycyloxytocin, sarcosyl or D-leucyl-oxytocin, or a mixture thereof. In some embodiments, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 IU of oxytocin is administered to an individual. In some embodiments, oxytocin is administered to an individual at a rate of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mU/min. In some embodiments, oxytocin is administered to an individual for 2, 3, or more consecutive days. In some embodiments, the individual is female. In some embodiments, the individual is monitored for symptoms of any of the diseases, syndromes, and/or disorders encompassed herein. In some embodiments, the symptoms comprise pain, abnormal gait, abnormal balance, abnormal activity level, abnormal heart rate, abnormal heart rate variability, depression, anxiety, abnormal motor function, or a combination thereof.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION

### I. Examples of Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

### II. General Embodiments

Embodiments of the disclosure concern treatment and/or prevention of Ehlers-Danlos Syndrome (EDS). EDS may comprise one or a group of inherited disorders that affect connective tissues. In some embodiments, the terms Ehlers-Danlos Syndrome, EDS, postural orthostatic tachycardia syndrome (POTS), and dysautonomia spectrum disorder may be used interchangeably, such as when referring to a disease, syndrome, or disorder being treated. In some embodiments, EDS comprises Hypermobile EDS. In some embodiments, EDS comprises vascular EDS. Symptoms of EDS may comprise overly flexible joints, joint instability, joint dislocations, joint subluxations, stretchy skin, fragile skin, delayed wound healing, abnormal scar formation, dehiscence of scars, pain (including chronic pain), POTS, gastroparesis, headaches, dysmenorrhea, dysautonomia, neuropathy, or a combination thereof. In certain embodiments, one or more therapeutic compositions are administered to an individual to treat, prevent, reduce one or more symptoms, or delay the onset of EDS. In certain embodiments, one or more therapeutic compositions are administered to an individual to reverse, slow-down, or stop the progression of EDS in the individual.

In some embodiments, an individual having, or suspected of having, EDS is administered a therapeutically effective amount of a composition. In some embodiments, the composition comprises oxytocin and/or an analogue of oxytocin and/or other full or partial agonists (peptide and non-peptide) for the oxytocin receptor. The analogue of oxytocin may comprise one or more of carbetocin, demoxytocin, Z-prolyl-D-leucine, 1-N-methylhemicystine-oxytocin, 1-D-hemicystineoxytocin, glycyl-oxytocin, leucyl-oxytocin, phenylalanyl-oxytocin, valyl-oxytocin, glutaminyl-oxytocin, prolyl-oxytocin, glycyl-glycyl-oxytocin, leucyl-leucyl-oxytocin, leucyl-glycyl-glycyloxytocin, sarcosyl-oxytocin, D-leucyl-oxytocin, [Se—Se]-oxytocin-OH. Agonists and partial agonists for oxytocin receptor may comprise TC-OT-39 and alike. In some embodiments, the composition comprises oxytocin metabolites that might be active other than through the oxytocin receptor, such as OT(4-9) and OT(5-9).

### III. Administration of Therapeutic Compositions

In some embodiments, approximately or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 IU of a therapeutic composition, including oxytocin and/or an oxytocin analogue is administered to an individual. The oxytocin or oxytocin analogue may be administered in 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 mL of a solution, such as a saline solution. The administration may occur over 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more minutes. In some embodiments, the administration of the therapeutic composition occurs once. In some embodiments, the administration of the therapeutic composition occurs more than once. One skilled in the art may determine the amount, the rate, and the number of administrations of the therapeutic composition depending on certain factors such as severity of the disease and/or symptoms, and/or the individual's response to the administration of the therapeutic composition.

The therapy provided herein may comprise administration of a combination of therapeutic agents, such as a first therapy and a second therapy. In some embodiments, the first therapy comprises oxytocin. In some embodiments, the second therapy comprises an oxytocin analogue. In some embodiments, oxytocin and another therapy, including an oxytocin analogue, may be delivered in any chronological order. In some embodiments, the second therapy comprises an analgesic and/or an anti-inflammatory composition, such as a nonsteroidal anti-inflammatory drug, and/or another hormone such as growth hormone. The therapies may be administered in any suitable manner known in the art. For example, the first and second treatment may be administered sequentially (at different times) or concurrently (at the same time). In some embodiments, the first and second treatments are administered in a separate composition and may or may not be delivered at the same time. In some embodiments, the first and second treatments are in the same composition. In some cases the first and second treatments are delivered at the same time but are not present in the same composition or formulation.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agent(s) of the disclosure may be administered by the same route of administration or by different routes of administration. In some embodiments, the therapeutic agent, such as oxytocin and/or an oxytocin analogue is administered intravenously, intramuscularly, subcutaneously, topically, orally, transdermally, intraperitoneally, intraorbitally, by implantation, by inhalation, intrathecally, intraventricularly, sublingually, or intranasally. In some embodiments, the therapeutic agent(s) are delivered via a pump, such as a subcutaneous pump, for example an insulin pump. Some of the therapies might be given in slow-release formulation. The appropriate dosage and route of administration may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In some embodiments, a therapeutically effective amount may include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the therapeutic agent is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In some embodiments, the effective dose of the therapeutic agent is one which can provide a plasma concentration of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 125, 150, 175, 200, 210, 225, 250, 275, 300, 310, 325, 350, 375, 400, or greater pg/mL following injection, including immediately after injection. In some embodiments, the effective dose of the therapeutic agent is one which can provide a plasma concentration of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 125, 150, 175, 200, 210, 225, 250, 275, 300, 310, 325, 350, 375, 400, or greater pg/mL sustained after injection. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM. Doses can be denoted as international units (IU) that can be converted to weight units. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein. In some embodiments, one dose is needed to achieve a different effect than a dose in a different embodiment.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 days, 1, 2, 3, or 4 weeks, or 1, 2, 3, 4, 5, 6, 7, 8, 9, 10, 11, or 12 month intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

A. Pharmaceutical Compositions

In certain aspects, the compositions or agents for use in the methods, such as oxytocin and/or an oxytocin analogue, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skin, one or more joints, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 mL and 0.5 mL, for example. In some embodiments, the pharmaceutical composition prepared for intranasal administration comprises one or more compositions for maintaining stability.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

It is contemplated that other agents may be used in combination with certain aspects of the present embodiments to improve the therapeutic efficacy of treatment.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I: Changes in Primary Fibroblasts from Patients with Hypermobile EDS

Data using primary fibroblasts from patients with hypermobile EDS revealed changes in oxytocin receptor expression in hypermobile patients when compared with healthy controls. Changes were identified both at the RNA and the protein levels. Oxytocin (OXT) is a nonapeptide synthesized in the paraventricular (PVN) and the supraoptic nuclei (SON) in the hypothalamus. OXT is an abundant neuropeptide that is mostly known for its importance during parturition and lactation. The use of oxytocin (IM/IV) to induce labor and to prevent postpartum hemorrhage is a standard of care. Multiple published studies suggest a significant role for oxytocin in behavior and pain. OXT function is mediated through postsynaptic receptors. These receptors are widely distributed in several CNS regions, including cortex, olfactory system, basal ganglia, limbic system, thalamus, hypothalamus, brain stem, and dorsal horn of the spinal cord.

The connection between oxytocin and pain was widely investigated both in humans and animal models. Rash, AguirreCamacho, and Campbell in 2014 published a systematic review of the literature (1950-2012) to assess the association between oxytocin and pain. They reported that oxytocin increased pain tolerance in 29 of 33 animal studies. The average effect reported by these studies was large (Cohen d=2.28) and effect persisted across central and peripheral modes of administration and type of noxious stimulus used (eg, heat, electric). Analgesic effects were strongest 20 to 30 minutes after exogenous administration and lasted for approximately 1 hour. The results suggest that oxytocin acts as an analgesic for acute pain in animals. Preliminary research with humans offers consistent evidence to suggest that oxytocin decreases pain sensitivity.

### Example II: Purpose and Objectives

Studies encompassed herein investigate the potential effect of IV oxytocin on chronic pain and its functional consequences in individuals with hypermobile Ehlers Danlos syndrome. Chronic pain may be assessed both by pain scales and by patient reported outcomes. Functional consequence of chronic musculoskeletal pain may be evaluated using digital body sensors to record impairment of gait and balance and/or activity level and heart rate.

Specific aspects of this disclosure evaluate effect of IV oxytocin on chronic pain in patients with hypermobile Ehlers Danlos syndrome. Certain embodiments evaluate chronic pain before and after treatment with IV oxytocin. Primary outcome measure 1) Change in the individual's subjective reported pain following treatment with placebo compared to the change in reported pain following treatment with oxytocin as assessed by the following questionnaires: Numerical pain rating scale, Brief Pain Inventory, and McGill Pain Questionnaire.

Secondary Outcome measures 1) Change in gait and balance studies performance following treatment with placebo compared to the change in gait and balance performance following treatment with oxytocin as an objective functional outcome of pain. 2) Change in reported signs of depression and anxiety following treatment with placebo compared to the change in the reported signs following treatment with oxytocin as assessed by Hospital anxiety and depression scale and State-Trait Anxiety Inventory questionnaires. 3) Change in heart rate, inter-beat-interval (IBI), and physical activity as measured by ACTIHEART device comparing data collected prior, during and after placebo and oxytocin treatments.

### Example III: Protocol Risks/Subjects

E1. Risk Category
Category 2: Research involving greater than minimal risk, but presenting the prospect of direct benefit to the individual subjects.
E2. Subjects
Gender: Non-pregnant Females
Age: Adult (18-64 yrs)

### Example IV. Design

The present disclosure includes a single-site, single-blind, fixed-sequence study to evaluate the effect of IV oxytocin on chronic pain in female adult patients with hypermobile EDS. Hypermobile EDS is more prevalent in females and the symptoms are usually more severe in females and more females come to medical attention. Oxytocin secretion in the body is dynamic and can be affected by multiple factors including the menstrual period. In order to have minimum variation between tested individuals, this study may be in females, all participants may be at the same stage of their menstrual cycles.

All participating individuals may be individuals affected with hypermobile-EDS (hEDS) with chronic moderate pain. Some embodiments may include two periods of treatment —one with placebo and one with oxytocin. Each period of treatment may include three consecutive daily infusions (placebo or oxytocin). Each participant may receive placebo infusion (normal saline) in the first treatment period and oxytocin infusion in the second treatment period. Each period may be preceded by 6 days of daily pain evaluation using pain-evaluation questionnaire (‘pre-infusion evaluation’). This period (prior to placebo or prior to oxytocin treatment) may start 1-3 days after the end of the menstrual cycle. Participants may be blinded to the order of treatment.

For each treatment period, participants may arrive to the study site for three daily consecutive visits, each may last for 3-4 hours. Response variables may be collected prior, during and after each infusion. Patients may fill out pain evaluation questionnaires pre and post infusion. Blood pressure and heart rate measurements may be taken prior, during and after the infusion. Blood samples for measurements of oxytocin levels and blood samples for future analysis may be collected prior and after the infusion. Response to treatment by questionnaire may be evaluated also during 6 days after each three infusion days (placebo or oxytocin).

Inclusion Criteria:

1) Premenopausal Females, Age >18 years 2) Clinical diagnosis of hypermobile EDS according to the 2017 criteria for hEDS 3) Menstrual periods (range from 8 to 42 days) 4) Pain level greater that pain score of 4 out of 10 on a Numeric Rating Scale in at least two of the following locations: back, neck, shoulders, elbows, wrist, hand joints, hips, knees, ankles, on most days over the 3 months preceding enrollment. 5) On a stable regimen for pain control without any expected increase in dose of pain medications during the study period. 6) All participants should have a negative urine pregnancy test and agree to use an acceptable method of contraception (abstinence or barrier methods).

Exclusion Criteria:

1) Known allergy to OXT or preservatives in the medication; 2) Pregnancy; 3) Lactation; 4) A confirmed clinical diagnosis of autoimmune disorders that lead to joint inflammation and joint pain such as SLE, RA, psoriatic arthritis, ankylosing spondylitis, scleroderma, and enteropathic arthritis; 5) History of known cardiac arrhythmias (except for asymptomatic sinus tachycardia and sinus bradycardia); 6) Heart rate persistently greater than 110 per minute or less than 50 per minute; 7) QTC of >450 ms from EKG (electrocardiogram) test; 8) Taking oral or other hormonal contraceptives; 9) Individuals with a clinical condition which, in the view of the investigator compromises safety; 10) Inability to complete gait analysis; 11) Participating in another interventional study.

### Example V: Procedure

This example of a procedure includes a single-blind, fixed-sequence study. Some embodiments may include two treatment periods. During the first period, placebo may be administered and during the second period, oxytocin may be administered. The participants may be blinded to the sequence of treatment assignment. Each treatment period may start 1-3 days after the menstrual period and may be divided to the following: 1) pre-infusion period of 6 days during which baseline pain and anxiety evaluation may be done, 2) infusion period that includes 3 consecutive days of infusions, 3) post-infusion evaluation period of 6 days during which post-treatment pain and anxiety may be evaluated.

Pain and anxiety evaluation may be done using the following questionnaires: 1) The Brief Pain Inventory (BPI), 2) Numerical Rating Scale (NRS), 3) McGill Pain Questionnaire, 4) Hospital Anxiety and Depression Scale (HAD), and 5) State-Trait Anxiety Inventory (STAI-trait part).

Pre-Screen: Eligibility screening of subjects may be done prior to study enrollment. Interested participants may be consented by phone to screen for eligibility purposes. Eligibility screening may involve: 1) medical record review, 2) collecting history of pain relevant to inclusion, 3) informing patients that pain medication regimens should continue without change during the two weeks prior to the study and throughout the study duration.

Screen Visit: If participants are deemed appropriate candidates for the study, they may come to the study site for a screening visit. During the screening visit, the following procedures may be done: 1) signed informed consent, 2) history and physical examination, 3) review of concomitant medications, 4) review of medical records, if available, 5) Urine pregnancy test may be administered, 6) EKG for assessment of QTc. QTc may be calculated by Fredericia formula, 7) enrollment checklist may be completed, and 8) ACTIHEART device may be given to the participant.

Treatment period 1 may start soon after the end of menstrual period for subjects. Treatment period 1 may consist of the following:

A. Pre-infusion Period 1 (may last six days prior to infusion): 1) Questionnaires: Subjects may be asked to complete pain evaluation questionnaires (BPI, NRS, and McGill). Anxiety evaluation may be completed by patients using HAD and STAItrait and state parts questionnaires. 2) During this period, subjects may have an in-person evaluation for motor function and mobility evaluation (gait and balance) conducted by trained research staff. In certain embodiments, a skilled artisan will use validated wearable technologies for assessing spatio-temporal parameters of gait and balance. 3) During this period, participants may be asked to wear ACTIHEART device (for heart rate and activity recording) during 3 days prior to the infusion, during the 3 infusion days, and in the three days after the infusion.

Details about the heart rate and activity recording device ACTIHEART: a chest-worn device using self-adhesive ECG electrodes that records heart rate, inter-beat-interval (IBI), and physical activity.

Details about the gait and balance assessments at iCAMP Lab are as follow:

Gait assessment: Gait performance may be assessed using validated body worn sensors (LegSys, Biosensics LLC, USA). The device uses five sensor modules respectively attached to right and left anterior shins, right and left anterior thighs, and posteriorly to the lower back. Based on the subject's height and using a two-link inverse pendulum model, the following spatio-temporal gait parameters may be estimated: velocity, stride length, stride time, double support, single support, strideto-stride variability, and gait initiation. In addition, the center of mass (COM) range of motion during walking may be calculated by using the data from the sensor attached to lower-back. Gait may be assessed over a distance of 20 meters under 2 conditions: 1) walking at habitual speed and 2) walking at maximum speed (fast walking).

Balance assessment: Balance may be quantified using validated body worn sensors (BalanSens, Biosensics LLC, USA). The system measures ankle and hip motion in three dimensions (3D), 2D COM sway as well as RCI in ML and AP directions. Balance may be assessed according to Romberg protocol during eyes-open and eyes-closed condition during double, semi-tandem, and full tandem stances.

B. Infusion period 1 (Placebo Infusion over three days): Participants may arrive for three consecutive day visits. 1) Subject may be instructed to eat a light, non-fatty breakfast. 2) Vital signs (blood pressure and heart rate), including weight and height may be collected prior to infusion. 3) Provide two questionnaires (BPI, NRS) for subjects to complete. 4) Nursing staff may establish IV access for infusion and blood collection. 5) Prior to infusion, blood samples may be obtained for oxytocin levels and for storage. 6) Placebo infusion (200 ml, 0.9% NaCl, over 40 minutes). Blood pressure and heart rate may be monitored during and after the infusion. 7) Blood sample (for oxytocin levels and storage) may be collected immediately after the infusion on day 1. 8) Blood sample (for oxytocin levels and/or for storage) may be collected 2 hours after infusion on days 1 and 3. 9) Administer both the NPI and STAI-state part questionnaires prior to subject's discharge. Subjects may be discharged 2 hours after the infusion.

C. Post Infusion Period 1—Pain & Anxiety Evaluation for 6 days: Participants may complete questionnaires over six days at the investigator's discretion: BPI, NRS, McGill on days 1, 3, and 5 after infusion and HAD and STAI-state and trait questionnaires on day 6 after infusion. Participants may continue to wear ACTIHEART device for three days after the completion of infusion.

Treatment period 2 may start at least 2 weeks after the completion of treatment period 1 and soon after the end of menstrual period for subjects. Treatment period 2 may consist of the following:

A. Pre-infusion Period 2 (may last six days prior to infusion): 1) Questionnaires: Subjects may be asked to complete pain evaluation questionnaires (BPI, NRS, and McGill). Anxiety evaluation may be completed by patients using HAD and STAItrait and state parts questionnaires. 2) During this period, subjects may have an in-person evaluation for motor function and mobility evaluation (gait and balance) conducted by trained research staff. In some embodiments, a skilled artisan will use validated wearable technologies for assessing spatio-temporal parameters of gait and balance. 3) During this period, participants may be asked to wear ACTIHEART device (for heart rate and activity recording) during 3 days prior to the infusion, during the 3 infusion days, and in the three days after the infusion.

B. Infusion period 2 (oxytocin Infusion over three days): Participants may arrive for three consecutive day visits. 1) Subject may be instructed to eat a light, non-fatty breakfast. 2) Vital signs (blood pressure and heart rate), including weight and height may be collected prior to infusion. 3) Provide two questionnaires (BPI, NRS) for subjects to complete. 4) Nursing staff may establish IV access for infusion and blood collection. 5) Prior to infusion, blood samples may be obtained for oxytocin levels and for storage. 6) Oxytocin infusion (200 ml, 0.9% NaCl with 1 IU of oxytocin, over 40 minutes*). Blood pressure and heart rate may be monitored during and after the infusion. 7) Blood sample (for oxytocin levels and storage) may be collected immediately after the infusion on day 1. 8) Blood sample (for oxytocin levels and/or for storage) may be collected 2 hours after infusion on days 1 and 3. 9) Administer both the NPI and STAI-state part questionnaires prior to subject's discharge. Subjects may be discharged 2 hours after the infusion.

C. Post Infusion Period 2—Pain & Anxiety Evaluation for 6 days: 1) Participants may complete questionnaires over six days at the investigator's discretion: BPI, NRS, McGill on days 1, 3, and 5 after infusion and HAD and STAI-state and trait questionnaires on day 6 after infusion. 2) Participants may continue to wear ACTIHEART device for three days after the completion of infusion. Participants may have an in-person evaluation for gait and balance studies during the post-infusion period. This motor function and mobility evaluation may be conducted by trained research staff. *Note: The dose oxytocin administration is based on the American College of Obstetricians and Gynecologists' (ACOG) Practice Bulletins for postpartum hemorrhage. From the bulletin: Many organizations have recommended active management of the third stage of labor as a method to reduce the incidence of postpartum hemorrhage. Prophylactic oxytocin, by dilute intravenous infusion (bolus dose of 10 units), or intramuscular injection (10 units), remains the most effective medication with the fewest adverse effects”. This clinical protocol allows the administration of bolus of oxytocin in a rate of 333 mU/min. A skilled artisan may administer only 1 unit of oxytocin over 40 minutes rate of 25 mU/min to avoid any adverse effects.

### Example VI: Data Analysis

Data Analysis

Specific aspects of this disclosure compare the change in pain scores between the pre and post placebo treatment period vs pre and post IV oxytocin treatment period. Changes from baseline to endpoint may be evaluated by paired T test Baseline and endpoint that may be compared: 1) Average pain and pain-effect scores (using the BPI) in the days prior to each infusion period (placebo and oxytocin) and average pain and pain-effect scores in the days after each infusion period. 2) Comparing the difference in average pain and pain-effect scores (using the BPI) pre and post infusion between placebo and oxytocin treatment. 3) Simple pain scale scores (NPRS) prior to infusion and simple pain scale scores 2 hours after the infusion on every day of infusion in both infusion periods (placebo and oxytocin). 4) Anxiety and depression scores prior and post infusion periods and comparing scores following placebo and oxytocin treatments. 5) Gait and Balance studies prior to first (placebo) infusion, between treatment periods and after the second infusion period (oxytocin). 6) Comparing heart rate, inter-beat-interval (IBI), and physical activity between data from three days prior to infusion (placebo or oxytocin), data from recorded during three days of infusion and data recorded during three days after infusion and comparing the data between placebo and oxytocin treatment. In certain embodiments, a skilled artisan compares heart rate, blood pressure with oxytocin infusion compared to levels with placebo. In certain embodiments, a skilled artisan compares oxytocin levels in the plasma prior to the first infusion, immediately after the first infusion and 2 hours after the first infusion for both placebo and oxytocin. P-values of 0.05 or less may be considered statistically significant

### Example VII: Considerations

Oxytocin secretion in the body is dynamic and can be affected by multiple factors including the menstrual period. In order to have minimum variation between tested individuals, certain methods may be conducted only in females, all participants may be at the same stage of their menstrual cycles. There does not appear to be any difference in the risk from using oxytocin in females vs males.

According to accepted guidelines, the maximum amount of blood to be drawn over a 24-hour period is 3% of total blood volume if the subject is an outpatient, and 5% of total blood volume if the subject is an inpatient. The maximum amount of blood which can be safely drawn from research participants in any one-month period should not exceed 10% of the total blood volume. All the participants in this study are adults. On average an adult have blood volume of approximately 5 liters. This may allow a skilled artisan to draw maximum of 150 ml per 24 hours and 500 ml over a total period of one month. The amount of blood planned to be drawn may be lower than the maximum allowed. A record of total blood volume withdrawn during each visit may be maintained in the subject's medical record. Blood drawn during each treatment period (in one month) include: Day 1: Oxytocin levels (10 mL) and storage blood (10 mL) at preinfusion, immediately post infusion, and 2 hours post infusion. This sums up to 60 ml per 24 hours. Day 3: Storage blood (10 mL) at preinfusion and 2 hours post infusion. This sums up to 20 ml per 24 hours. Total of 80 ml per one month.

### Example XIII: Sample Collection

Sample: Blood

All participants are adults. Blood may be collected on days 1 and 3 during each treatment period. The two treatment periods are at least one month apart. On each treatment period, blood may be drawn prior to treatment, immediately after treatment and 2 hours after the end of the infusion on day 1 and prior to treatment and 2 hours after the treatment on day 3. Visit 1 and 3 in treatment period 1—blood draw may include in each visit: Day 1—Pre-treatment: 10 ml (2 teaspoons) for oxytocin measurements, 10 ml (2 teaspoons) for storage. immediately after treatment: 10 ml (2 teaspoons) for oxytocin measurements, 10 ml (2 teaspoons) for storage. Two hours after treatment: 10 ml (2 teaspoons) for oxytocin measurements, 10 ml (2 teaspoons) for storage. Total of 60 ml of blood for this day (12 teaspoons). Day 3—Pre-treatment: 10 ml (2 teaspoons) for storage. Two hours after treatment: 10 ml (2 teaspoons) for storage. Total of 20 ml of blood for this day (4 teaspoons). Total blood collection during treatment period 1-80 ml (16 teaspoons) Visit 1 and 3 in treatment period 2—blood draw may include in each visit: Day 1—Pre-treatment: 10 ml (2 teaspoons) for oxytocin measurements, 10 ml (2 teaspoons) for storage. immediately after treatment: 10 ml (2 teaspoons) for oxytocin measurements, 10 ml (2 teaspoons) for storage. Two hours after treatment: 10 ml (2 teaspoons) for oxytocin measurements, 10 ml (2 teaspoons) for storage. Total of 60 ml of blood for this day (12 teaspoons). Day 3—Pre-treatment: 10 ml (2 teaspoons) for storage. Two hours after treatment: 10 ml (2 teaspoons) for storage. Total of 20 ml of blood for this day (4 teaspoons). Total blood collection during treatment period 1-80 ml (16 teaspoons) Samples for oxytocin measurements may be obtained on ice and plasma may be stored immediately. Each sample for storage may be stored half (5 ml) as plasma and half (5 ml) as serum.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating Hypermobile Ehlers-Danlos syndrome, Dysautonomia spectrum disorder, and/or postural orthostatic tachycardia syndrome, comprising the step of administering to an individual in need thereof an effective amount of oxytocin or a functionally active derivative or analogue thereof.

2. The method of claim 1, wherein the functionally active derivative or analogue thereof is carbetocin, demoxytocin, Z-prolyl-D-leucine, 1-N-methylhemicystine-oxytocin, 1-D-hemicystineoxytocin, glycyl-, leucyl-, phenylalanyl-, prolyl-, glycyl-glycyl-, leucyl-leucyl, or leucyl-glycyl-glycyloxytocin, sarcosyl or D-leucyl-oxytocin, or a mixture thereof.

3. The method of claim 1, wherein Hypermobile Ehlers-Danlos syndrome comprises Ehlers-Danlos syndrome type III.

4. The method of claim 1, wherein the effective amount of oxytocin comprises 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 IU of oxytocin.

5. The method of claim 1, wherein the step of administering oxytocin comprises administering oxytocin at a rate of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mU/min.

6. The method of claim 1, wherein the step of administering oxytocin comprises administration of oxytocin for 2, 3, or more consecutive days.

7. The method of claim 1, wherein the individual is a female.

8. The method of claim 1, further comprising the step of monitoring symptoms in the individual.

9. The method of claim 8, wherein the step of monitoring symptoms is before, during, and/or after the step of administering the effective amount of oxytocin.

10. The method of claim 8, wherein the symptoms comprise pain, abnormal gait, abnormal balance, abnormal activity level, abnormal heart rate, abnormal heart rate variability, depression, anxiety, abnormal motor function, or a combination thereof.
